(19) United States
(12) Patent Application Publication
(10) Pub. No.: US 2010/0233739 A1
(43) Pub. Date: Sep. 16, 2010

(54) USE OF URINARY NGAL TO DIAGNOSE UNILATERAL AND BILATERAL URINARY OBSTRUCTION

(76) Inventors: Jonathan BARASCH, New York, NY (US); Thomas L. NICKOLAS, New York, NY (US)

Correspondence Address: WilmerHale/Columbia University, 399 PARK AVENUE, NEW YORK, NY, 10022, US

(21) Appl. No.: 12/704,875

(22) Filed: Feb. 12, 2010

Related U.S. Application Data
(60) Provisional application No. 61/151,998, filed on Feb. 12, 2009.

(51) Int. Cl. G01N 33/53; G01N 33/00

(52) U.S. Cl. 435/7.92; 436/86

(57) ABSTRACT

In one embodiment, the present invention is directed to methods for diagnosis of urinary tract obstruction (UTO), and to methods for distinguishing between unilateral and bilateral UTO. In some aspects, the diagnostic methods of the invention are based on determining whether a bodily fluid sample, such as a urine sample, contains an amount of NGAL protein that exceeds or is less than a certain threshold level, or that falls within a certain range. The present invention also provides diagnostic kits for the diagnosis of UTO and for distinguishing between unilateral and bilateral UTO.

This application claims the benefit of the filing date of U.S. Provisional Patent Application No. 61/151,998, filed Feb. 12, 2009, the contents of which are hereby incorporated by reference.

All patents, patent applications and publications cited herein are hereby incorporated by reference in their entirety.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

Bilateral and unilateral urinary obstruction may be difficult to diagnose in patients given the insensitivity of the current diagnostic marker, serum creatinine (SCr), especially in cases of unilateral kidney obstruction, where SCr is an especially weak marker. But urinary obstruction may cause tubular damage in the kidneys within hours. Therefore there is a need for a more sensitive diagnostic biomarker of both bilateral and unilateral urinary obstruction, so that the condition can be diagnosed sufficiently early so that early tubular damage may be mitigated and promptly treated.

### SUMMARY OF THE INVENTION

The present invention is based, in part, on certain discoveries which are described more fully in the Examples section of the present application. For example, the present invention is based, in part, on the discovery that levels of NGAL protein in the urine of patients with urinary tract obstruction (“UTO”) are higher than the levels of NGAL in the urine of control patients, and on the discovery that the level of NGAL protein in the urine is higher in patients with bilateral UTO (“bUTO”) as compared to patients with unilateral UTO (“uUTO”). It is also a discovery of the present invention that measuring NGAL levels is far superior to measuring serum creatinine (“SCr”) levels for the purposes of diagnosing UTO, including uUTO and bUTO. Thus, the present invention provides diagnostic methods for diagnosis of UTO, uUTO, and bUTO, and for distinguishing between uUTO and bUTO, and compositions and kits for use in such diagnostic methods.

In one embodiment, the present invention provides a method for determining whether a subject has urinary tract obstruction (UTO), the method comprising determining the concentration of NGAL protein in a urine sample from a subject, wherein a concentration of NGAL in the urine sample that exceeds a threshold amount indicates that the subject has UTO, and wherein a concentration of NGAL in the urine sample that is less than the threshold amount indicates that the subject does not have UTO. In one embodiment, the threshold amount is between about 30 micrograms per gram creatinine and about 120 micrograms per gram creatinine. In one embodiment, the determining step comprises performing an immunoassay, such as an ELISA, to detect NGAL protein. In some embodiments, the methods further comprise adjusting the subject's treatment regimen based on whether the concentration of NGAL in the urine sample exceeds or is less than the threshold amount.

In another embodiment, the present invention provides a method for determining whether a subject with urinary tract obstruction has unilateral urinary tract obstruction (uUTO) or bilateral urinary tract obstruction (bUTO), the method comprising determining the concentration of NGAL protein in a urine sample from a subject, wherein a concentration of NGAL in the urine sample that exceeds a threshold amount indicates that the subject has bUTO, and wherein a concentration of NGAL in the urine sample that is less than the threshold amount indicates that the subject has uUTO. In one embodiment, the threshold is from about 130 micrograms per gram creatinine to about 400 micrograms per gram creatinine. In one embodiment, the determining step comprises performing an immunoassay, such as an ELISA, to detect NGAL protein. In some embodiments, the methods further comprise adjusting the subject's treatment regimen based on whether the concentration of NGAL in the urine sample exceeds or is less than the threshold amount.

In another embodiment, the present invention provides diagnostic kits for determining whether a subject has a UTO, such kits comprising, for example: a device for detecting NGAL protein in the urine; a positive control containing NGAL protein; and instructions indicating threshold levels of NGAL above which a diagnosis of UTO can be made. In one embodiment, the diagnostic kits contain instructions indicating that NGAL levels of above about 30 micrograms per gram creatinine to above about 120 micrograms per gram creatinine can be used to make a diagnosis of UTO. In another embodiment, the diagnostic kits contain instructions indicating that NGAL levels of above about 130 micrograms per gram creatinine to above about 400 micrograms per gram creatinine can be used to make a diagnosis of bilateral UTO (bUTO). In one embodiment, the device in the diagnostic kits comprises an anti-NGAL antibody. In one embodiment, the device in the diagnostic kits comprises an ELISA plate, a urine dipstick, or a test strip.

These and other embodiments of the invention are further described in the following sections of the application, including the Detailed Description, Examples, Claims, and Drawings.

### BRIEF DESCRIPTION OF THE FIGURES

FIG. 1. Distribution of serum creatinine (“initial crt”) among matched controls and cases of obstruction. Serum creatinine levels among controls (indicated as “0”) and cases of unilateral obstruction (indicated as “1”) were not significantly different. In comparison to controls, serum creatinine was significantly elevated in among cases of bilateral obstruction (indicated as “2”) (p<0.01).

FIG. 2. Distribution of uNGAL among matched controls and cases of obstruction. One patient with bilateral obstruction had an uNGAL of 2623 mg/g creatinine, not shown. uNGAL levels among cases of unilateral obstruction were 8-fold greater than among controls (p<0.0001). uNGAL levels among cases of bilateral obstruction were 26-fold greater than among controls (p<0.0001).

FIG. 3. Receiver operator characteristics and area under curve (AUC) values for uNGAL and serum creatinine.

### DETAILED DESCRIPTION

The present invention is based, in part, on certain discoveries which are described more fully in the Examples section of the present application. For example, the present invention is based, in part, on the discovery that levels of NGAL protein in the urine of patients with urinary tract obstruction (“UTO”) are higher than the levels of NGAL in the urine of control patients, and on the discovery that the level of NGAL protein in the urine is significantly higher in patients with bilateral UTO (“bUTO”) as compared to patients with unilateral UTO (“uUTO”). It is also a discovery of the present invention that measuring NGAL levels is far superior to measuring serum creatinine (“SCr”) levels for the purposes of diagnosing UTO, including uUTO and bUTO. Thus, the present invention provides diagnostic methods for diagnosis of UTO, uUTO, and bUTO, and for distinguishing between uUTO and bUTO, and compositions and kits for use in such diagnostic methods.

### ABBREVIATIONS AND DEFINITIONS

The abbreviation “NGAL” refers to Neutrophil Gelatinase Associated Lipocalin. NGAL is also referred to in the art as human neutrophil lipocalin, siderocalin, a-micropglobulin related protein, Scn-NGAL, lipocalin 2, 24p3, superinducible protein 24 (SIP24), uterocalin, and neu-related lipocalin. These alternative names for NGAL may be used interchangeably herein. Unless stated otherwise, the term “NGAL”, as used herein, includes any NGAL protein, fragment, or mutant that is expressed in the kidney, and which can be detected in a bodily fluid such as urine. In some embodiments the NGAL protein is wild-type human NGAL.

The abbreviation “uNGAL” is an abbreviation for urinary NGAL and refers to NGAL in the urine.

The abbreviation “UTO” refers to urinary tract obstruction.

The abbreviation “uUTO” refers to unilateral urinary tract obstruction.

The abbreviation “bUTO” refers to bilateral urinary tract obstruction.

The abbreviation “CKD” refers to chronic kidney disease.

The abbreviation “AKI” refers to acute kidney injury.

The abbreviation “SCr” refers to serum creatinine.

The abbreviation “ESRD” refers to end-stage renal disease.

The abbreviation “GFR” refers to glomerular filtration rate.

The abbreviation “eGFR” refers to estimated glomerular filtration rate.

The abbreviation “MDRD” refers to modification of diet in renal disease The MDRD equation for calcuting GFR and/or eGFR is well known in the art. See, for example, Levey et al., (1999), “A more accurate method to estimate glomerular filtration rate from serum creatinine: a new prediction equation. Modification of Diet in Renal Disease Study Group.” Ann. Intern. Med. 1999; 130: 461-470, and Levey et al., (2000), “A simplified equation to predict glomerular filtration rate from serum creatinine” J. Am. Soc. Nephrol. 2000; (11):155A, the contents of which are hereby incorporated by reference.

The abbreviation “ROC” refers to receiver operating characteristic. ROC curves are widely used in the art for assessing diagnostic and prognostic tests. See, for example, Zweig & Campbell, (1993), “Receiver-operating characteristic (ROC) plots: a fundamental evaluation tool in clinical medicine”. Clinical chemistry 39 (8): 561-577; and Zou et al., (2007). “Receiver-operating characteristic analysis for evaluating diagnostic tests and predictive models.” Circulation, 6; 115(5): 654-7; and Lasko et al., (2005), “The use of receiver operating characteristic curves in biomedical informatics.” Journal of Biomedical Informatics, 38(5):404-415, the contents of each which are hereby incorporated by reference.

The abbreviation “AUC” refers to area under the curve, such as the area under an ROC curve.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### DESCRIPTION

In one aspect of the invention, levels of NGAL protein in a bodily fluid, such as urine, that exceed a certain threshold amount can be used to diagnose urinary tract obstruction (UTO), including both unilateral and bilateral UTO, and to distinguish between unilateral UTO and bilateral UTO. It is a discovery of the invention that, in the two studies performed (see Example 1 (study 1) and Example 2 (study 2)), control patients had mean uNGAL concentrations of 16 (study 1) and 16.4 (study 2) μg/g creatinine, while patients with uUTO had mean uNGAL concentrations of 50 (study 1) and 134.7 (study 2) μg/g creatinine, and patients with bUTO had mean uNGAL concentrations of 209 (study 1) and 428.4 (study 2) μg/g creatinine.

Accordingly, in one embodiment the present invention provides methods for determining whether a subject has UTO, the methods comprising measuring the amount of NGAL protein in a bodily fluid, such as urine, from the subject, wherein an amount of NGAL protein that exceeds a threshold level, such as a threshold level of about 30 μg/g creatinine, or about 40 μg/g creatinine, or about 50 μg/g creatinine, or about 60 μg/g creatinine, or about 70 μg/g creatinine, or about 80 μg/g creatinine, or about 90 μg/g creatinine, or about 100 μg/g creatinine, or about 110 μg/g creatinine, or about 120 μg/g creatinine, indicates that the subject has a UTO. Conversely, an amount of NGAL protein that is less than the threshold level can indicate that the subject does not have UTO.

In another embodiment the present invention provides methods for determining whether a subject has bUTO, the methods comprising measuring the amount of NGAL protein in a bodily fluid, such as urine, from the subject, wherein an amount of NGAL protein that exceeds a threshold level, such as a threshold level of about 130 μg/g creatinine, or about 140 μg/g creatinine, or about 150 μg/g creatinine, or about 160 μg/g creatinine, or about 170 μg/g creatinine, or about 180 μg/g creatinine, or about 190 μg/g creatinine, or about 200 μg/g creatinine, or about 225 μg/g creatinine, or about 250 μg/g creatinine, or about 275 μg/g creatinine, or about 300 μg/g creatinine, or about 350 μg/g creatinine, or about 400 μg/g creatinine indicates that the subject has bUTO. Conversely, an amount of NGAL protein that is less than the threshold level can indicate that the subject does not have bUTO.

In another embodiment, the present invention provides methods for distinguishing between uUTO and bUTO in a subject, such as subject known to have UTO. Such methods comprise measuring the amount of NGAL protein in a bodily fluid, such as urine, from the subject, wherein an amount of NGAL protein that exceeds a threshold level, such as a threshold level of about 130 μg/g creatinine, or about 140 μg/g creatinine, or about 150 μg/g creatinine, or about 160 μg/g creatinine, or about 170 μg/g creatinine, or about 180 μg/g creatinine, or about 190 μg/g creatinine, or about 200 μg/g creatinine, or about 225 μg/g creatinine, or about 250 μg/g creatinine, or about 275 μg/g creatinine, or about 300 μg/g creatinine, or about 350 μg/g creatinine, or about 400 μg/g creatinine indicates that the subject has bUTO. Conversely, an amount of NGAL protein that is less than the threshold level can indicate that the subject does not have bUTO, but may have uUTO.

In one embodiment the above methods can be used for the early detection of UTO for example, before the onset of symptoms of UTO. Accordingly, in one aspect, the above methods be used to diagnose UTO in a subject who is not exhibiting signs of UTO.

In another embodiment, the present invention provides a method for monitoring the progression of UTO in a subject, the method comprising measuring the amount of NGAL protein in a first bodily fluid sample taken from the subject and a second bodily fluid sample that is taken from the subject at a later period in time, wherein an amount of NGAL protein in the second sample that exceeds the amount of NGAL protein in the first sample, indicates that the UTO is worsening, and an amount of NGAL protein in the second sample that is less than the amount of NGAL protein in the first sample, indicates that the UTO is improving. In one embodiment, the first sample can be taken before the initiation of therapy for UTO, and the second sample can be taken after the initiation of such therapy. In another embodiment, both samples can be taken after the initiation of therapy. Thus, such methods can be used to monitor the effect of therapy on the progression of UTO in a subject.

In yet another embodiment, the present invention provides a solution to the problem of determining whether a subject is a candidate for treatment of UTO, the method comprising measuring the amount of NGAL protein in a bodily fluid, such as urine, from the subject, wherein an amount of NGAL protein that exceeds a threshold level indicates that the subject has is a candidate for treatment of UTO. Conversely an amount of NGAL protein that is less than the threshold level can indicate that the subject is not a candidate for treatment of UTO. In other embodiments, such methods also comprise subsequently treating the subject.

Another aspect of the invention provides a method of monitoring the effectiveness of a treatment for UTO in a subject, the method comprising the steps of: i) obtaining a baseline sample of a body fluid, such as urine, from the subject, ii) determining the level of NGAL in the baseline sample; iii) providing at least one treatment for the UTO iv) obtaining at least one post-treatment sample of the body fluid from the subject; v) determining the level of NGAL in the post-treatment sample; and vi) evaluating the effectiveness of the treatment, based on comparing the level of NGAL in the post-treatment sample to the level of NGAL in the baseline sample.

It should be noted that in all of the embodiments above that deal with making an assessment relating to UTO based on detecting a level of NGAL in the urine that exceeds a threshold amount, ranges of uNGAL amounts can be used in the place of threshold values. For example, the threshold amounts provided above can be substituted with ranges.

For example, uUTO may be indicated by an amount of uNGAL that falls within the range of about 30 μg/g-200 μg/g creatinine, or about 40 μg/g creatinine-200 μg/g creatinine, or about 50 μg/g creatinine-200 μg/g creatinine, or about 60 μg/g creatinine-200 μg/g creatinine, or about 70 μg/g creatinine-200 μg/g creatinine, or about 80 μg/g creatinine-200 μg/g creatinine, or about 90 μg/g creatinine-200 μg/g creatinine, or about 100 μg/g creatinine-200 μg/g creatinine, or about 110 μg/g creatinine-200 μg/g creatinine. Also, the upper end of each of the preceding ranges can be adjusted, for example to about 210 μg/g creatinine, or about 220 μg/g creatinine, or about 230 μg/g creatinine, or about 240 μg/g creatinine, or about 250 μg/g creatinine, or about 275 μg/g creatinine, or about 300 μg/g creatinine.

Similarly, bUTO may be indicated by an amount of uNGAL that falls within the range of about 130 μg/g creatinine-700 μg/g creatinine, or about 140 μg/g creatinine-700 μg/g creatinine, or about 150 μg/g creatinine-700 μg/g creatinine, or about 160 μg/g creatinine-700 μg/g creatinine, or about 170 μg/g creatinine-700 μg/g creatinine, or about 180 μg/g creatinine-700 μg/g creatinine, or about 190 μg/g creatinine-700 μg/g creatinine, or about 200 μg/g creatinine-700 μg/g creatinine, or about 225 μg/g creatinine-700 μg/g creatinine, or about 250 μg/g creatinine-700 μg/g creatinine, or about 275 μg/g creatinine-700 μg/g creatinine, or about 300 μg/g creatinine-700 μg/g creatinine, or about 350 μg/g creatinine-700 μg/g creatinine, or about 400 μg/g creatinine-700 μg/g creatinine. Also, the upper end of each of the preceding ranges can be adjusted, for example to about 750 μg/g creatinine, or about 800 μg/g creatinine, or about 850 μg/g creatinine, or about 900 μg/g creatinine, or about 950 μg/g creatinine, or about 1000 μg/g creatinine.

The diagnostic methods described herein can be combined in various ways. Furthermore, the following description applies to all of the diagnostic methods described herein.

All of the diagnostic methods of the invention, such as those described above, can comprise one or more additional steps. The diagnostic methods of the invention may comprise one or more steps for obtaining the bodily fluid sample from the subject, for example using the methods described herein. The diagnostic methods of the invention may comprise one or more steps for treating the bodily fluid sample from the subject, for example using the methods described herein. The diagnostic methods of the invention may comprise one or more steps for detecting and/or measuring NGAL levels in the bodily fluid sample, for example using the methods described herein. The diagnostic methods of the invention may comprise one or more steps for treating the subject or altering the subject's treatment based on the level of NGAL detected and/or whether the measured NGAL level is greater or less than the chosen cut-off level. For example, if the subject's NGAL level exceeds a cut-off level for diagnosing UTO (e.g. uUTO or bUTO), the subject may be treated for UTO.

According to the methods of the invention, such as the diagnostic methods described above, the bodily fluid can be any sample in which NGAL can be detected, including, but not limited to, blood, serum, or urine. In some embodiments the bodily fluid is urine.

Also according to the methods of the invention, the subject can be any animal that is susceptible to UTO. In some embodiments the subjects are rodents, such as mice. In some embodiments, the subjects are cows, pigs, sheep, goats, cats, horses, dogs, and/or any other species of animal used as livestock or kept as pets. In some embodiments the subjects are human subjects. In some embodiments, the subjects are already suspected to have UTO before testing according to the methods of the invention.

In certain embodiments, the NGAL protein detected and/or measured in the methods of the present invention has an amino acid sequence as defined by one of the following GenBank accession numbers, NP 005555 (human NGAL), CAA67574 (human NGAL), P80188 (human NGAL), AAB26529 (human NGAL), P11672 (mouse NGAL), P30152 (rat NGAL), AAI132070 (mouse NGAL), AAI132072 (mouse NGAL), AAH33089 (human NGAL), and CAA58127 (human NGAL), or is a homolog, variant, derivative, fragment, or mutant thereof, and/or has at least 80% sequence identity, e.g., 85%, 90%, 95%, 98% or 99% sequence identity, with one of the above sequences.

In certain embodiments of the invention, it can be desirable to use a positive control for the detection of NGAL. NGAL protein for use as a positive control can be obtained from any source or produced by any method known in the art. For example, NGAL protein can be recombinantly produced. Methods for the recombinant production of proteins are well known in the art. For example, a nucleotide sequence encoding NGAL can be included in an expression vector containing expression control sequences and expressed in, and purified from, any suitable cell type, such as bacterial cells or mammalian cells. For example, for use as a positive control in the methods of the invention, recombinant NGAL can be produced as described in Yang, et al. (2002) Mol Cell 10, 1045-1056; Goetz et al. (2002) Mol. Cell 10, 1033-1043; Goetz et al. (2000) Biochemistry 39, 1935-1941; and Mori, et al. (2005) J. Clin Invest. 115, 610-621, the contents of which are hereby incorporated by reference.

As described above, in certain embodiments, the present invention provides methods for determining whether a subject has UTO, and methods for distinguishing between uUTO and bUTO, such methods comprising measuring the amount of NGAL protein in a bodily fluid, such as urine, from the subject, wherein an amount of NGAL protein that exceeds a threshold level indicates that the subject has UTO (either uUTO or bUTO). In addition to the threshold levels specified herein, a threshold level can also be selected by reviewing the data provided in the Examples section of this application, so that the threshold level is sufficiently high that it is more likely than not that a subject having that level of NGAL will have a UTO, or will have a uUTO as opposed to a bUTO, or vice versa.

It should also be noted that, although the amounts of NGAL described herein are generally referred to in terms of the amount by mass of NGAL relative to the amount by mass of urine creatinine (UCr), e.g. NGAL μg/g creatinine, NGAL can also be measured and/or represented in other units, including, but not limited to measurements of the amount of NGAL by mass (e.g. in nanograms or micrograms), or measurements of the amount of NGAL by concentration (e.g. in ng/mL), or any other units, and it should be understood that amounts of NGAL measured and/or represented in other units can be equivalent to the amounts and ranges described herein in terms of μg/g creatinine. The present invention is not limited to methods that comprise measuring NGAL and also measuring creatinine and/or to methods that comprise calculating the amount of NGAL in a sample of bodily fluid in terms of the number of micrograms per gram of creatinine. For example, an amount of NGAL that is represented herein as 100 μg/g creatinine, can also be represented in terms of, and encompasses, alternative measurements/units that correspond to the same amount of NGAL, e.g. the same amount of NGAL expressed in terms of mass (e.g. ng), or in terms of concentration (e.g. ng/mL) or in any other units. One of skill in the art can readily make the necessary conversions between units.

Furthermore, it should be noted that threshold levels or ranges of NGAL other than those specifically described herein may be used in accordance with the invention. It is a discovery of the invention that NGAL levels are higher in the urine of subjects with UTO as compared to subjects without UTO, and are higher in subjects with bUTO as compared to subjects with bUTO. The mean levels of uNGAL in such groups (control, uUTO, and bUTO groups) may vary in different groups of subjects or depending on the methodology used to measure NGAL levels. Accordingly, the present invention provides for the general concept of using uNGAL levels to diagnose UTO and to distinguish uUTO from bUTO, and not only methods that rely on the specific thresholds and ranges provided herein.

In certain embodiments, other biomarkers can be assessed in addition to NGAL in order to determine whether a subject has UTO. For example, the present invention provides that, in addition to having a high level of urinary NGAL, UTO patients can also have one or more of: (i) high urea nitrogen and/or (ii) a high serum creatinine level.

According to the methods of the invention, samples of a bodily fluid can be obtained and/or tested using any means. For example, methods for collecting, handling and processing urine, blood, serum and plasma, and other body fluids, are well known in the art and can be used in the practice of the present invention. In some embodiments, two or more consecutive or subsequent samples of a body fluid can be taken. Depending upon the circumstances, including the level of NGAL in a sample and the clinical condition of the subject, the subject's body fluid can be sampled daily, or weekly, or within a few weeks, or monthly or within a few months, semi-annually, annually, or within several years, and at any interval in between. Repeat sampling can be done at a period of time after treatment to detect any change in UTO status. Sampling need not be continuous, but can be intermittent (e.g., sporadic). In some embodiments, it is not necessary to obtain and keep a sample of the bodily fluid from the subject. For example, in some embodiments, the subject can urinate onto a test strip, for example a test strip of the type used in pregnancy testing kits. In other embodiments, a sample of bodily fluid, such as blood from a pin prick, can be applied onto a test strip—for example a test strip similar to those used for blood typing.

Although generally the sample of a bodily fluid, such as blood or urine, is obtained from a subject and tested by a laboratory or by a medical professional (for example using an automated urinalysis machine configured to test for NGAL, or an nNGAL testing kit, e.g. a urine dipstick based kit, or an ELISA based kit), home-testing kits are also within the scope of the present invention. In one aspect, the present invention comprises a kit for performing the methods of the invention, containing, for example, a device for detecting NGAL protein in the urine, and optionally including a positive control containing NGAL protein, and optionally including instructions, for example regarding the threshold levels of NGAL above which a diagnosis of UTO, uUTO, or bUTO, can be made. The device in such kits can comprise, for example, an ELISA plate, a dipstick or a test strip to be dipped in a urine sample or to have a sample or urine applied thereto, or a stick on which the subject should urinate. In some embodiments, such devices are configured such that they give a positive result only if the level of NGAL exceeds a threshold level, such as one of the threshold levels described herein. Methods for making and using such devices are well known in the art. Kits (ELISA kits), antibodies, and other reagents for detection of NGAL in the urine are commercially available, e.g. from Bioporto Diagnostics A/S and from R & D Systems, and can be used to make a kit according to the present invention. Such kits can be used by subjects themselves (e.g. home testing kits) or can be used by medical or laboratory staff.

The present invention also provides methods based on measuring the levels of circulating NGAL, as opposed to urinary NGAL. Blood sampling is a routine clinical procedure, and blood samples of individuals may have been stored and preserved, providing a valuable database of historical samples that may be used to predict the progression disease in certain patients.

According to the methods of the invention, the presence and/or amount of NGAL protein in a bodily fluid, such as urine, can be detected and/or measured using any means known in the art. For example, in one embodiment, NGAL protein can be detected using antibodies that are specific to NGAL. Any antibody, such as a monoclonal or polyclonal antibody, that binds to NGAL can be used. For example, monoclonal antibodies that bind to NGAL are described in “Characterization of two ELISAs for NGAL, a newly described lipocalin in human neutrophils”, Lars Kjeldsen et al., (1996) Journal of Immunological Methods, Vol. 198, 155-16, the contents of which are herein incorporated by reference. An example of a polyclonal antibody for NGAL is described in “An Iron Delivery Pathway Mediated by a Lipocalin”, Jun Yang et al., Molecular Cell, (2002), Vol. 10, 1045-1056, herein incorporated by reference in its entirety. To prepare this polyclonal antibody, rabbits were immunized with recombinant gel-filtered NGAL protein. Sera were incubated with GST-Sepharose 4B beads to remove contaminants, yielding the polyclonal antibodies in serum, as described by the applicants in Jun Yang et al., Molecular Cell (2002). Further non-limiting examples of antibodies that can be used to detect NGAL protein in the methods of the invention are also provided in the Examples. Antibodies that bind to NGAL are also available commercially, for example from the Antibody Shop, Copenhagen, Denmark, as HYB-211-01, HYB-211-02, and NYB-211-05. In addition, one of skill in the art can readily produce antibodies that bind to NGAL, or can have them produced by an antibody production company.

Any method can be used to detect and or measure the levels of NGAL protein, including, but not limited to, immunohistochemistry-based methods, immuno-blotting based methods, immunoprecipitation-based methods, affinity-column based methods (including immunoaffinity column based methods), ELISA-based methods, other methods in which an NGAL antibody is immobilized on a solid substrate (such as beads), and the like. In some such methods the antibody to NGAL, or a secondary or tertiary antibody that binds directly or indirectly to the NGAL antibody, can be labeled with a detectable moiety, such as a fluorescent moiety, a radioactive moiety, or a moiety that is an enzyme substrate and can be used to generate a detectable moiety, such as horse radish peroxidase. Such methods are well known in the art and can be used to detect the presence and/or measure the amount of NGAL in a bodily fluid sample, such as urine, without undue experimentation.

In circumstances where the amount of NGAL is to be measured, positive controls containing known amounts of NGAL protein can be used, for example for calibration purposes. NGAL protein for use as a positive control can be obtained from any source or produced by any method known in the art. For example, NGAL protein can be recombinantly produced. Methods for the recombinant production of proteins are well known in the art. For example, a nucleotide sequence encoding NGAL can be included in an expression vector containing expression control sequences and expressed in, and purified from, any suitable cell type, such as bacterial cells or mammalian cells. For example, for use as a positive control in the methods of the invention, recombinant NGAL can be produced as described in Yang, et al. (2002) Mol Cell 10, 1045-1056; Goetz et al. (2002) Mol. Cell 10, 1033-1043; Goetz et al. (2000) Biochemistry 39, 1935-1941; and Mori, et al. (2005) J. Clin Invest. 115, 610-621, the contents of which are hereby incorporated by reference.

In other aspects of the invention, a diagnosis of UTO, such as uUTO or bUTO, can be based upon, or can include, detecting the presence of NGAL protein or mRNA in tissues, such as in tissues of the urinary tract or the kidney, as opposed to in a bodily fluid such as urine, for example by detecting a high level of NGAL protein or mRNA, or by detecting a specific localization of NGAL protein or mRNA. Such methods can be used alone, or can be used in conjunction with one or more other methods, such as the methods described herein for detection of NGAL in urine or other bodily fluids or standard diagnostic methods based on the examination of biopsy samples, etc. Methods for assessing the expression and/or localization of NGAL protein or mRNA in tissues of the urinary tract or in the kidney in situ are also provided by the invention, for example methods wherein, for example, labeled agents that bind to NGAL protein or mRNA are delivered to a subject and can be visualized in vivo, for example using imaging techniques such as CAT scan-based techniques and MRI-based techniques.

Detection of NGAL mRNA or protein can be determined using standard techniques and methodologies known to those of skill in the art, for example using samples obtained by biopsy. For example, NGAL mRNA can be detected by in situ hybridization using probes specific for NGAL, or by any other method known to be useful for detection of specific mRNAs, including, but not limited to, PCR-based techniques. The sequence of NGAL, including human NGAL, is known in the art. Similarly, sequences of probes and primers that can be used to detect NGAL are known in the art. In addition, NGAL protein can be detected using antibodies that are specific to NGAL, e.g. monoclonal or polyclonal antibodies can be used. In addition, detection methods that can be used, include, but are not limited to, immunohistochemistry-based methods and the like. Antibodies that are specific to NGAL and that could be used to detect NGAL in the kidneys are known in the art. Monoclonal antibodies for NGAL, are described, for example, in “Characterization of two ELISAs for NGAL, a newly described lipocalin in human neutrophils”, Lars Kjeldsen et al., (1996) Journal of Immunological Methods, Vol. 198, 155-16, herein incorporated by reference in its entirety. Non-limiting examples of antibodies that can be used to detect NGAL protein are provided in the Examples. Antibodies that bind to NGAL are also available commercially, for example from the Antibody Shop, Copenhagen, Denmark, as HYB-211-01, HYB-211-02, and NYB-211-05. Typically, HYB-211-01 and HYB-211-02 can be used with NGAL in both its reduced and unreduced forms. An example of a polyclonal antibody for NGAL is described in “An Iron Delivery Pathway Mediated by a Lipocalin”, Jun Yang et al., Molecular Cell, (2002), Vol. 10, 1045-1056, herein incorporated by reference in its entirety. To prepare this polyclonal antibody, rabbits were immunized with recombinant gel-filtered NGAL protein. Sera were incubated with GST-Sepharose 4B beads to remove contaminants, yielding the polyclonal antibodies in serum, as described by the applicants in Jun Yang et al., Molecular Cell (2002).

Those skilled in the art will recognize, or be able to ascertain using no more than routine experimentation, many equivalents to the specific embodiments of the invention described herein. Such equivalents are intended to be within the scope of the present invention.

The invention is further described by the following non-limiting Examples.

### EXAMPLES

### Example 1

Observational data from a human cohort revealed that uNGAL is elevated in patients with AKI due to urinary obstruction. A case control study of patients with unilateral and bilateral obstruction was conducted to better describe the characteristics of uNGAL in these conditions and to correlate uNGAL with kidney function.

Consecutive patients presenting to the emergency department who donated a urine sample were enrolled in the study. After prospective review of hospital course, laboratory and radiographic studies, patients with urinary obstruction were identified. Obstruction was defines as either radiographically (hydronephrosis or hydroureter) or clinically (presence of post-void residual urine volume>500 cc). Patients with AKI not related to obstruction were excluded from the study. Cases (obstruction) were age-, gender- and race-matched to controls who had similar baseline kidney function. Kidney function was determined by Modification of Diet in Renal Disease glomerular filtration rate (MDRD GFR). NGAL was measured by immunoblot and urine creatinine (UCr) was measured by spectrophotometry. uNGAL levels were corrected for UCr and all values expressed as (m/g). SCr values expressed in mg/dl.

Twenty-four patients with obstruction were matched to twenty-four controls. Patient characteristics are shown in Table 1. Groups were well matched for age, race, sex and baseline SCr. Patients with obstruction had significantly elevated mean SCr and uNGAL at presentation p<0.05 and p<0.001 respectively. Patients with bilateral obstruction had higher uNGAL than patients with unilateral obstruction, mean uNGAL (SD) 209 (207) versus 50 (44) p<0.01. Both uNGAL and SCr were significantly elevated in patients with bilateral obstruction as compared to matched controls 209 (207) vs 16 (12) p<0.001 for uNGAL and 2.9 (2.8) vs 1.3 (0.6) p=0.04 for SCr. However, in unilateral obstruction, only uNGAL was significantly elevated compared to matched controls 50 (44) versus 16 (11) p=0.007, while there was no significant difference between presenting SCr among these groups 1.0 (0.3) vs. 0.8 (0.2) p=0.1. The AUC (95% CI) for uNGAL and SCr for the diagnosis of urinary obstruction was 0.872 (0.767-0.976) and 0.67 (0.517-0.823) respectively. Table 1 below shows patient characteristics and uNGAL means from the study.

This Example shows that uNGAL can identify urinary obstruction with a higher degree of sensitivity and specificity than SCr. uNGAL is elevated in both unilateral and bilateral urinary obstruction while serum creatinine is only significantly elevated in bilateral obstruction.

TABLE 1

| | Obstructed | |
|---|---|---|
| | Patients | Controls |
| Age | 60 (22) | 60 (20) |
| % Female | 42 | 42 |
| % Black | 13 | 13 |
| Baseline SCr | 1.1 (0.6) | 1.0 (0.5) |
| Admission SCr | 1.9 (2.2) | 1.0 (0.5)* |
| Mean uNGAL μg/g | 130 (167) | 16 (11)^{†} |
| creatinine | | |

*p-value < 0.5,
^{†}p-value < 0.001

### Example 2

UTO may be difficult to diagnose given that serum creatinine (SCr) may not rise, especially in cases of unilateral obstruction, urine output is unreliable indicator as it can be normal or even increased in UTO, and pain is typically only present in acute cases. Complete or prolonged partial UTO can lead to tubular atrophy and eventually irreversible renal injury, with animal models showing significant nephron loss after 24 hours of obstruction (1).

Neutrophil gelatinase-associated lipocalin (NGAL) is a 22 kD protein produced by the nephron in response to tubular epithelial damage. NGAL is a marker of tubulointerstitial injury and has been shown to be massively upregulated in AKI. Urine NGAL (uNGAL) was previously shown to be elevated in patients with acute kidney injury (AKI) in a large cohort of patients presenting to an urban Emergency Department (2).

In the present study it was demonstrated that NGAL is elevated in cases of AKI due to UTO. A case-control study was performed as part of a cross-sectional study evaluating relationships between novel biomarkers and kidney function. Patient selection for the study was as follows. Adult subjects over age 18 undergoing hospital admission were included in the study. Patients already receiving renal replacement therapy were excluded. Cases of UTO were matched by age, race, gender, and baseline kidney function to control subjects.

Potential cases were identified by review of the chief complaint and admission diagnosis. Unilateral obstruction cases were identified on the basis of obstruction of one ureter using radiographic evidence of hydronephrosis or hydroureter. Bilateral obstruction cases were identified at either the level of the bladder or urethra with retention of >500 cc of urine with or without hydronephrosis on radiographic imaging. Subjects who had stable renal function during the course of their hospitalization were used as controls.

2,162 patients were recruited during hospital admission. 70 potential cases of UTO were identified. Electronic medical records were reviewed to determine actual cases of obstruction. 16 patients had bilateral obstruction and 25 had unilateral obstruction. Patient characteristics, uNGAL and serum creatinine levels, and renal ultrasound frequency stratified by degree of obstruction, are illustrated in Table 2 below.

As demonstrated in Table 3 below, uNGAL was found to be a significantly better discriminator of obstruction than serum creatinine as determined by area under the curve (AUC) analysis. Serum creatinine was shown to be a poor discriminator of obstruction as the AUC was near 0.5. Table 4 below shows the clinical outcomes of the patients in the study stratified by degree of obstruction.

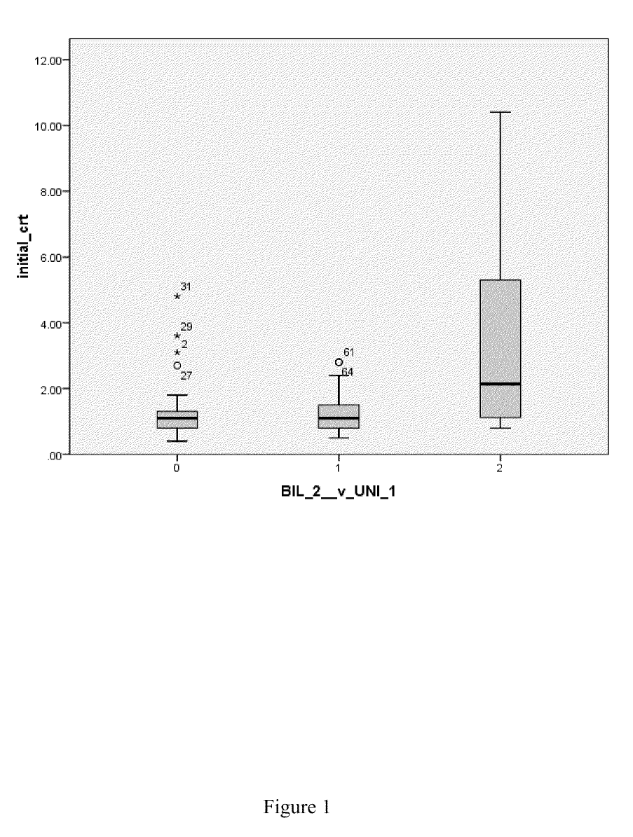

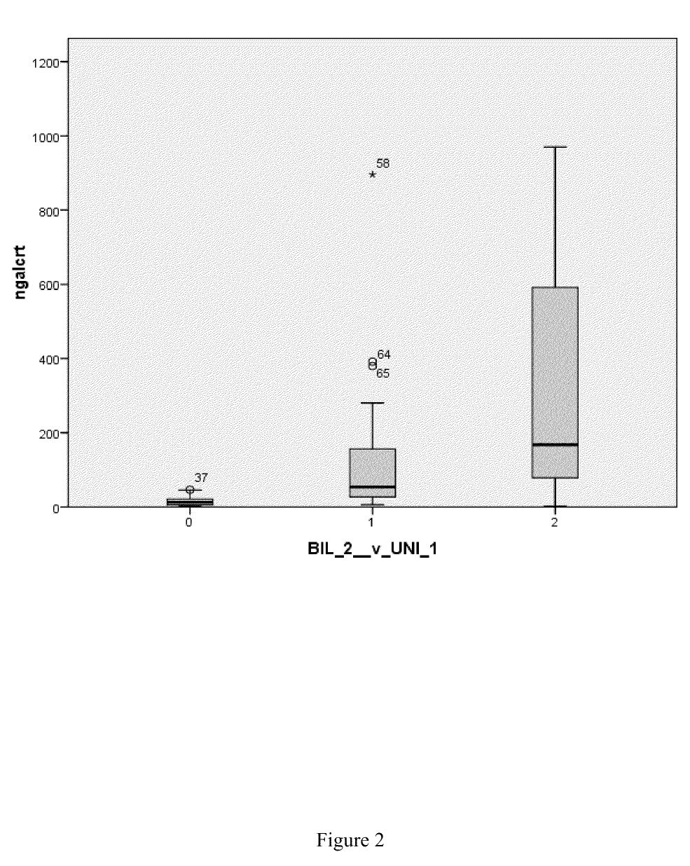

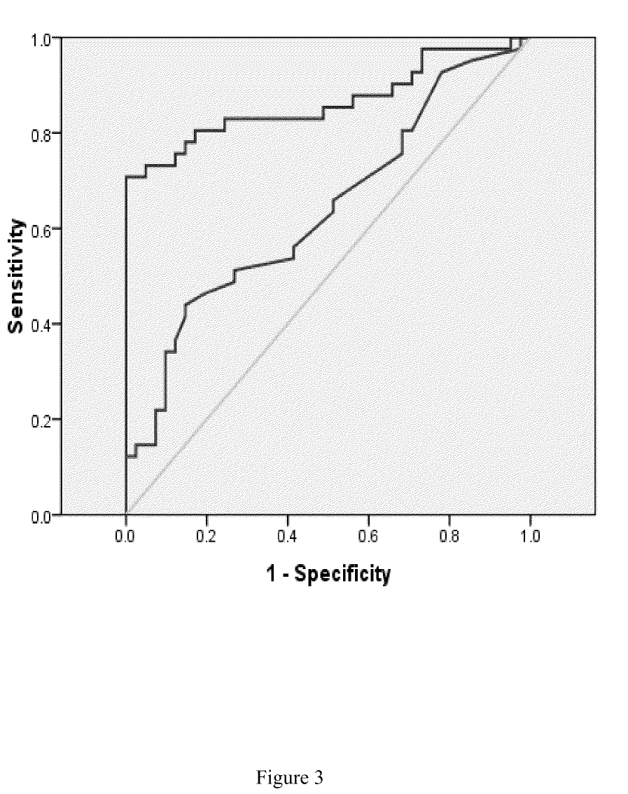

FIGS. 1 to 3 further illustrate some of the data from this study. FIG. 1 illustrates the distribution of serum creatinine (“initial crt”) among matched controls and cases of obstruction. Serum creatinine levels among controls (indicated as “0”) and cases of unilateral obstruction (indicated as “1”) were not significantly different. In comparison to controls, serum creatinine was significantly elevated in among cases of bilateral obstruction (indicated as “2”) (p<0.01). FIG. 2 illustrates the distribution of uNGAL among matched controls and cases of obstruction. One patient with bilateral obstruction had an uNGAL of 2623 mg/g creatinine, not shown. uNGAL levels among cases of unilateral obstruction were 8-fold greater than among controls (p<0.0001). uNGAL levels among cases of bilateral obstruction were 26-fold greater than among controls (p<0.0001). FIG. 3 shows receiver operator characteristic and area under the curve (AUC) analysis for uNGAL and serum creatinine.

The data presented herein shows that NGAL expression is induced in bilateral and unilateral urinary obstruction and can be measured in the urine, and that NGAL is significantly elevated in unilateral UTO despite normal serum creatinine levels. uNGAL levels among cases of unilateral obstruction were found to be 8-fold greater than among controls (p<0.0001). uNGAL levels among cases of bilateral obstruction were found to be 26-fold greater than among controls (p<0.0001). Thus NGAL levels are higher in bilateral UTO as compared to unilateral obstruction. This may be because both kidneys are affected and produce NGAL. In the present study the area under the curve (AUC) for uNGAL as a discriminator of obstruction was found to be superior to that of serum creatinine. In fact serum creatinine measurements were found to be insufficient to indicate UTO, having an AUC close to 0.5.

TABLE 2

| | | | Unilateral | Bilateral |
|---|---|---|---|---|
| | All Patients | | Obstruction | Obstruction |
| | (n = 82) | Controls (n = 41) | (n = 25) | (n = 16) |
| Age (yrs) | 58.7 (20.3) | 58.7 (19.6) | 51.7 (21.8)‡ | 69.8 (15.3)* |
| Female (%) | 38.1 | 39 | 60‡ | 6.2* |
| Black (%) | 9.5 | 9.8 | 8 | 12.5 |
| Baseline GFR | 80.3 (30.5) | 79.7 (32.4) | 90.7 (26.5) | 69.3 (26.9) |
| (mL/min/m2) | | | | |
| Baseline SCr | 1.1 (0.5) | 1.1 (0.5) | 0.9 (0.3)‡ | 1.3 (0.6) |
| (mg/dl) | | | | |
| ER Presentation GFR | 67.5 (37.1) | 77.6 (37.2) | 67.4 (31.7)† | 41.9 (33.7)** |
| ER Serum Creatinine | 1.7 (1.8) | 1.3 (0.9) | 1.3 (0.7)‡ | 3.5 (3.2)** |
| (mg/dl) | | | | |
| NGAL (μg/g | 132.9 (338.5) | 16.4 (13.3) | 134.7 (194.2)** | 428.4 (654.0)** |
| creatinine) | | | | |

*p < 0.05 compared to controls,
**p < 0.01 compared to controls,
†p < 0.05 compared to bilateral obstruction,
‡p < 0.01 compared to bilateral obstruction

TABLE 3

| Marker | AUC and 95% CI |
|---|---|
| Serum Creatinine | 0.640 (0.519-0.760) |
| (mg/dl) | |
| NGAL | 0.864 (0.779-0.950) |
| (μg/g creatinine) | |

TABLE 4

| | | | Unilateral | Bilateral |
|---|---|---|---|---|
| | All Patients | Controls | Obstruction | Obstruction |
| | (n = 82) | (n = 41) | (n = 25) | (n = 16) |
| Length of Stay | 6.1 (5.1) | 5.7 (4.4) | 4.5 (3.2)† | 9.5 (7.7) |
| (days) | | | | |
| Renal Consult | 13.1 | 2.4 | 18.2*‡ | 37.5** |
| (%) | | | | |
| In Hospital | 1.3 | 0 | 0 | 6.2 |
| Mortality (%) | | | | |
| Ureteric Stenting | 10.7 | 0 | 20‡ | 25 |
| (%) | | | | |

†p < .05 compared to bilateral obstruction,
‡p < .01 compared to bilateral obstruction,
*p < .05 compared to controls,
**p < .01 compared to controls

### REFERENCES

(1) Bander, S J et al. Long-term effects of 24-hour unilateral obstruction on renal function in the rat. Kidney Int 1985; 28:614.
(2) Nickolas, T L et al. Sensitivity and Specificity of a Single emergency department measurement of urinary neutrophil gelatinase-associated lipocalin for diagnosing acute kidney injury. Ann Intern Med. 2008 Jun. 3; 148(11):810-9

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention.

What is claimed is:

1. A method for determining whether a subject has urinary tract obstruction (UTO), the method comprising determining the concentration of NGAL protein in a urine sample from a subject, wherein a concentration of NGAL in the urine sample that exceeds a threshold amount indicates that the subject has UTO, and wherein a concentration of NGAL in the urine sample that is less than the threshold amount indicates that the subject does not have UTO.

2. The method of claim 1, wherein the threshold amount is between about 30 micrograms per gram creatinine and about 120 micrograms per gram creatinine.

3. The method of claim 1, wherein the determining step comprises performing an immunoassay to detect NGAL protein.

4. The method of claim 3, wherein the immunoassay is an ELISA.

5. The method of claim 1, further comprising adjusting the subject's treatment regimen based on whether the concentration of NGAL in the urine sample exceeds or is less than the threshold amount.

6. The method of claim 1, wherein the subject is a human.

7. A method for determining whether a subject with urinary tract obstruction has unilateral urinary tract obstruction (uUTO) or bilateral urinary tract obstruction (bUTO), the method comprising determining the concentration of NGAL protein in a urine sample from a subject, wherein a concentration of NGAL in the urine sample that exceeds a threshold amount indicates that the subject has bUTO, and wherein a concentration of NGAL in the urine sample that is less than the threshold amount indicates that the subject has uUTO.

8. The method of claim 7, wherein the threshold is from about 130 micrograms per gram creatinine to about 400 micrograms per gram creatinine.

9. The method of claim 7, wherein the determining step comprises performing an immunoassay to detect NGAL protein.

10. The method of claim 9, wherein the immunoassay is an ELISA.

11. The method of claim 7, further comprising adjusting the subject's treatment regimen based on whether the concentration of NGAL in the urine sample exceeds or is less than the threshold amount.

12. The method of claim 7, wherein the subject is a human.

13. A diagnostic kit for determining whether a subject has urinary tract obstruction (UTO), the kit comprising: (a) a device for detecting NGAL protein in the urine; (b) a positive control containing NGAL protein; and (c) instructions indicating a threshold level of NGAL above which a diagnosis of UTO can be made.

14. The diagnostic kit of claim 13, wherein the threshold level is between about 30 micrograms per gram creatinine and about 120 micrograms per gram creatinine.

15. The diagnostic kit of claim 13, wherein the device for detecting NGAL protein in the urine comprises an anti-NGAL antibody.

16. The diagnostic kit of claim 13, wherein the device for detecting NGAL protein in the urine is an ELISA plate, a urine dipstick, or a test strip.

17. The diagnostic kit of claim 13, wherein the instructions indicate a threshold levels of NGAL above which a diagnosis of bilateral UTO can be made.

18. The diagnostic kit of claim 17, wherein the threshold level is between about 130 micrograms per gram creatinine and about 400 micrograms per gram creatinine.
